(19) United States
(12) Patent Application Publication
(10) Pub. No.: US 2025/0086206 A1
(43) Pub. Date: Mar. 13, 2025

(54) INFORMATION PROCESSING APPARATUS, INFORMATION PROCESSING METHOD, AND STORAGE MEDIUM

(71) Applicant: NEC Corporation, Minato-ku, Tokyo (JP)

(72) Inventor: Masafumi Oyamada, Tokyo (JP)

(73) Assignee: NEC Corporation, Minato-ku, Tokyo (JP)

(21) Appl. No.: 18/285,976

(22) PCT Filed: May 12, 2023
(86) PCT No.: PCT/JP2023/017860

§ 371 Date: Oct. 6, 2023

(51) Int. Cl. G06F 16/332; G06F 40/30; G16H 80/00

(52) U.S. Cl. CPC G06F 16/3329; G06F 40/30; G16H 80/00

(57) ABSTRACT

To facilitate determination of the reliability of output of a language model, provided is an information processing apparatus (1) including: an acquisition section (11) that acquires a target text; a generation section (12) that generates a text corresponding to the target text acquired by the acquisition section (11) with use of a language model trained to generate a text based on an input text; a query generation section (13) that generates a query from the text generated by the generation section (12); an extraction section (14) that extracts a document related to the query from a database in a retrieval process using the query; and an output section (15) that outputs a result obtained by adding information identifying the document to the text generated by the generation section (12).

### TECHNICAL FIELD

The present invention relates to an information processing apparatus, an information processing method, and a program, all of which carry out language processing on text.

### BACKGROUND ART

Techniques that use language models are known. The language models are trained by using text data and are configured to carry out language processing. Patent Literature 1 discloses a question response apparatus that generates a response to a question sentence based on entities extracted from the question sentence and a value of the perplexity of a language model calculated from the question sentence, the model assuming question sentences of a specific field. The question response apparatus divides the text of the question sentence into morphemes, which are the smallest units having meanings, that is, words. Then, the apparatus issues a request for keyword search to an external search engine by using nouns included in the words as keywords. Further, the question response apparatus uses a response from the external search engine as the answer to the input question sentence.

### CITATION LIST

### Patent Literature

[Patent Literature 1]
Japanese Patent Application Publication Tokukai No. 2015-87796

### SUMMARY OF INVENTION

### Technical Problem

The question response apparatus disclosed in Patent Literature 1 is not configured to verify whether the response from the external search engine is correct. Thus, the user cannot determine whether the response to the question sentence is correct.

An example aspect of the present invention has been made in view of the above problem, and an example object thereof is to provide a technique for facilitating determination of the reliability of output of a language model.

### Solution to Problem

An information processing apparatus in accordance with an example aspect of the present invention includes: acquisition means for acquiring a target text; generation means for generating a text corresponding to the acquired target text with use of a language model trained to generate a text based on an input text; query generation means for generating a query from the text generated by the generation means; extraction means for extracting a document related to the query from a database in a retrieval process using the query; and output means for outputting a result obtained by adding information identifying the document to the text generated by the generation means.

An information processing method in accordance with an example aspect of the present invention includes: acquiring, by at least one processor, a target text; generating, by the at least one processor, a text corresponding to the target text with use of a language model trained to generate a text based on an input text; generating, by the at least one processor, a query from the generated text; extracting, by the at least one processor, a document related to the query from a database in a retrieval process using the query; and outputting, by the at least one processor, a result obtained by adding information identifying the document to the generated text.

A program in accordance with an example aspect of the present invention causes a computer to carry out: an acquisition process of acquiring a target text; a generation process of generating a text corresponding to the target text with use of a language model trained to generate a text based on an input text; a query generation process of generating a query from the text generated in the generation process; an extraction process of extracting a document related to the query from a database in a retrieval process using the query; and an output process of outputting a result obtained by adding information identifying the document to the text generated in the generation process.

### Advantageous Effects of Invention

According to an example aspect of the present invention, it is possible to facilitate determination of the reliability of output of a language model.

### BRIEF DESCRIPTION OF DRAWINGS

FIG. 1 is a block diagram illustrating the configuration of an information processing apparatus in accordance with a first example embodiment.

FIG. 2 is a flowchart illustrating the flow of an information processing method in accordance with the first example embodiment.

FIG. 3 is a block diagram illustrating the configuration of an information processing apparatus in accordance with a second example embodiment.

FIG. 4 is a flowchart illustrating the flow of an information processing method in accordance with the second example embodiment.

FIG. 5 is a diagram illustrating an example of each process carried out by the information processing apparatus in accordance with the second example embodiment.

FIG. 6 is a diagram illustrating a specific example of a second result outputted from the information processing apparatus in accordance with the second example embodiment.

FIG. 7 is a block diagram illustrating the configuration of an information processing system in accordance with a third example embodiment.

FIG. 8 is a table showing an example of documents in the third example embodiment of the present invention.

FIG. 9 is a table showing an example of information on authors of the documents in the third example embodiment of the present invention.

FIG. 10 is a flowchart illustrating the flow of an information processing method in accordance with the third example embodiment of the present invention.

FIG. 11 is a diagram illustrating an example of images displayed on an information processing terminal in accordance with the third example embodiment of the present invention.

FIG. 12 is a block diagram illustrating an example of the hardware configuration of the information processing apparatus in accordance with each of the example embodiments of the present invention.

### EXAMPLE EMBODIMENTS

### First Example Embodiment

The following description will discuss a first example embodiment of the present invention in detail with reference to the drawings. The present example embodiment is a basic form of example embodiments described later.

### (Configuration of Information Processing Apparatus)

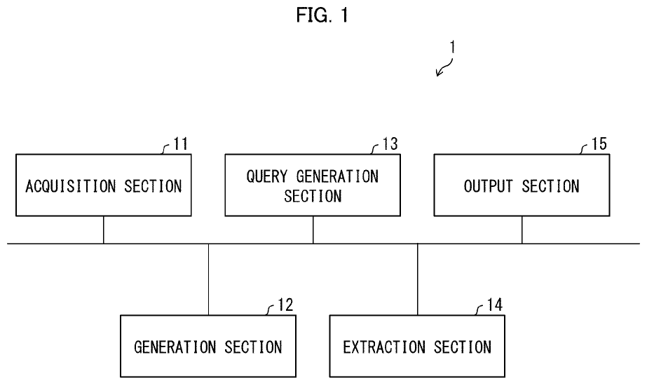

The following description will discuss the configuration of an information processing apparatus 1 in accordance with the present example embodiment with reference to FIG. 1. FIG. 1 is a block diagram illustrating the configuration of the information processing apparatus 1. The information processing apparatus 1 includes an acquisition section 11 (acquisition means), a generation section 12 (generation means), a query generation section 13 (query generation means), an extraction section 14 (extraction means), and an output section 15 (output means).

The acquisition section 11 acquires a target text. The generation section 12 generates a text corresponding to the target text acquired by the acquisition section 11, with use of a language model trained to generate a text based on an input text. The query generation section 13 generates a query from the text generated by the generation section 12. The extraction section 14 extracts a document related to the query from a database in a retrieval process using the query. The output section 15 outputs a result obtained by adding information identifying the document to the text generated by the generation section 12.

As described in the foregoing, the information processing apparatus 1 in accordance with the present example embodiment employs a configuration including: the acquisition section 11 that acquires a target text; the generation section 12 that generates a text corresponding to the target text acquired by the acquisition section 11, with use of a language model trained to generate a text based on an input text; the query generation section 13 that generates a query from the text generated by the generation section 12; the extraction section 14 that extracts a document related to the query from a database in a retrieval process using the query; and the output section 15 that outputs a result obtained by adding information identifying the document to the text generated by the generation section 12. Therefore, with the information processing apparatus 1 in accordance with the present example embodiment, it is possible to achieve an example advantage of facilitating determination of the reliability of output of the language model.

### (Program)

The abovementioned functions of the information processing apparatus 1 may be realized by a program. The program in accordance with the present example embodiment causes a computer to carry out: an acquisition process of acquiring a target text; a generation process of generating a text corresponding to the target text with use of a language model trained to generate a text based on an input text; a query generation process of generating a query from the text generated in the generation process; an extraction process of extracting a document related to the query from a database in a retrieval process using the query; and an output process of outputting a result obtained by adding information identifying the document to the text generated in the generation process. With this program, it is possible to facilitate determination of the reliability of output of the language model.

### (Flow of Information Processing Method)

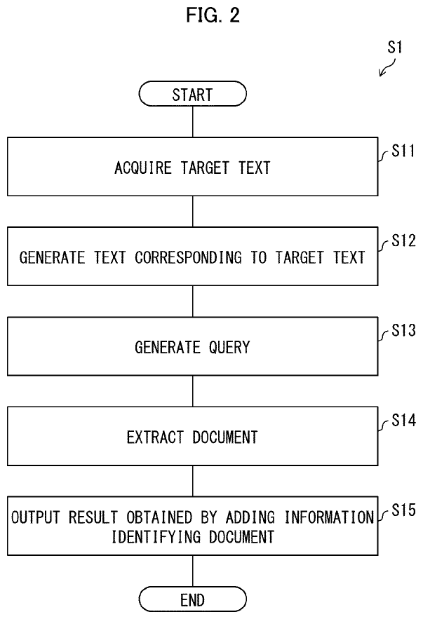

The following description will discuss the flow of an information processing method S1 in accordance with the present example embodiment with reference to FIG. 2. FIG. 2 is a flowchart illustrating the flow of the information processing method S1. It should be noted that steps of this information processing method may be carried out by a processor included in the information processing apparatus 1 or by a processor of another apparatus.

In S11, at least one processor acquires a target text. In S12, the at least one processor generates a text corresponding to the target text with use of a language model trained to generate a text based on an input text. In S13, the at least one processor generates a query from the text generated in S12. In S14, the at least one processor extracts a document related to the query from a database in a retrieval process using the query. In S15, the at least one processor outputs a result obtained by adding information identifying the document to the text generated in S12.

As described in the foregoing, the information processing method S1 in accordance with the present example embodiment includes: acquiring, by at least one processor, a target text; generating, by the at least one processor, a text corresponding to the target text with use of a language model trained to generate a text based on an input text; generating, by the at least one processor, a query from the generated text; extracting, by the at least one processor, a document related to the query from a database in a retrieval process using the query; and outputting, by the at least one processor, a result obtained by adding information identifying the document to the generated text. Therefore, with the information processing method S1 in accordance with the present example embodiment, it is possible to achieve an example advantage of facilitating determination of the reliability of output of the language model.

### Second Example Embodiment

The following description will discuss a second example embodiment of the present invention in detail with reference to the drawings. The same reference numerals are given to constituent elements which have functions identical to those described in the first example embodiment, and descriptions as to such constituent elements are omitted as appropriate.

### (Outline of Information Processing Apparatus 2)

An information processing apparatus 2 in accordance with the present example embodiment is an apparatus that acquires a target text and outputs a result (text etc.) obtained by carrying out language processing on the target text. Further, to the result outputted from the information processing apparatus 2, information identifying a document related to the result is added. The document will be described later. In the following description, the “target text” and the “text” are also referred to as the “string”.

The information processing apparatus 2 carries out language processing with use of a language model M trained by using text data. The language model M receives, as input, a string (text) and outputs a result obtained by carrying out language processing on the string. The result outputted from the language model M may be a string or alternatively, may be an image. The language processing carried out by the language model M is not particularly limited. Examples of the language model M may include: a process of generating a text based on an input text; text classification; emotion analysis; information extraction; text summarization; text generation; image generation; and question answering.

The following description will discuss the language model M in detail. The language model M is created by means of learning of the relationship between words in a text (text data), and is a model that generates, from the target string, a related string related to the target string. With use of the language model that has been made to learn statements and texts of various contexts, it is possible to generate the related string of reasonable contents related to the target string.

Examples of the language model M may include, but not limited to: large language models (LLMs) such as Bidirectional Encoder Representations from Transformers (BERT), Generative Pre-trained Transformer (GPT), Text-to-Text Transfer Transformer (T5), Robustly optimized BERT approach (ROBERTa), Efficiently Learning an Encoder that Classifies Token Replacements Accurately (ELECTRA); and learning models created by transfer learning or fine tuning with use of a pre-trained model (e.g., Chat Generative Pre-trained Transformer, ChatGPT).

Further, the string generated by the language model M is not limited to a natural language. The language model M may output an artificial language (a program source code or the like) for, for example, a string inputted in a natural language. For example, the language model M receives, as the target string, input of a question “how to acquire data including a specific string from the database?”. For the input, the language model M may output a program source code for carrying out database processing. Alternatively, the language model M may output a natural language corresponding to the string inputted in an artificial language.

The string acquired by the information processing apparatus 2 is not particularly limited, and may be, for example, a string including at least one word and an instruction indicating which language processing is to be carried out. For example, it is assumed that the information processing apparatus 2 acquires a string “what is the main business field of XX Corporation?”. In this case, the string includes five words, that is, “what is”, “the main”, “business field”, “of”, and “XX Corporation”; and an instruction to respond to the question stating “what is the main business field of XX Corporation?”.

### (Configuration of Information Processing Apparatus 2)

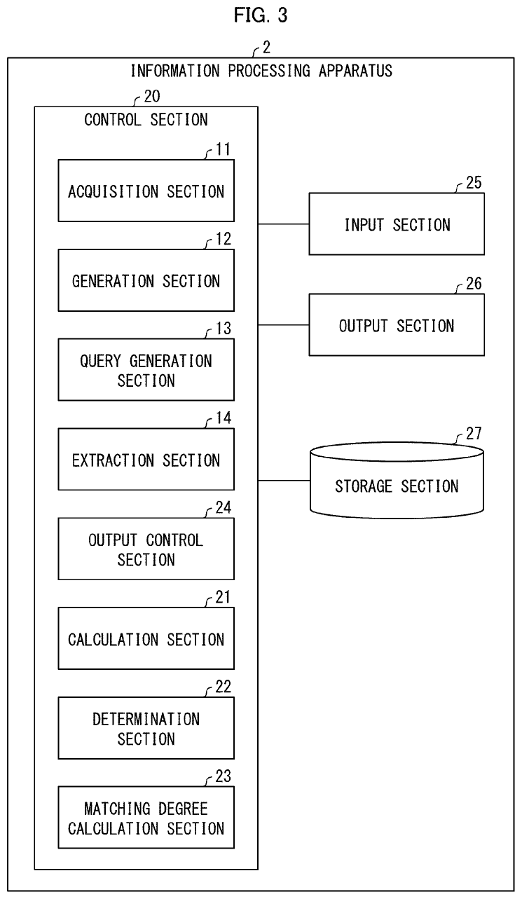

The following description will discuss the configuration of the information processing apparatus 2 with reference to FIG. 3. FIG. 3 is a block diagram illustrating the configuration of the information processing apparatus 2 in accordance with the present example embodiment. As illustrated in FIG. 3, the information processing apparatus 2 includes a control section 20, an input section 25, an output section 26, and a storage section 27.

The input section 25 is an interface for receiving input from a user. As an example, the input section 25 may provide information indicating the received user input to the control section 20. Examples of the input section 25 may include, but not limited to, a mouse, a keyboard, a touch pad, and a microphone.

The output section 26 is an interface for outputting data. As an example, the output section 26 may be an interface for outputting data to another apparatus connected thereto. In this case, for example, the output section 26 outputs a result outputted from the control section 20, to the another apparatus connected. As another example, the output section 26 may be a display device that displays an image or may be a speaker that outputs sound. When the output section 26 is the display device, the output section 26 displays an image including the result outputted from the control section 20.

The storage section 27 stores data referred to by the control section 20. An example of the data stored in the storage section 27 may be documents. Examples of the storage section 27 may include, but not limited to, a flash memory, a hard disk drive (HDD), a solid state drive (SSD), and a combination thereof.

The storage section 27 also stores the language model M. It should be noted that “the storage section 27 stores the language model M” means that the storage section 27 stores parameters defining the language model M. The language model M may be stored in another storage device (e.g., an external server) other than the storage section 27.

The control section 20 controls constituent elements included in the information processing apparatus 2. Further, as illustrated in FIG. 3, the control section 20 includes an acquisition section 11, a generation section 12, a query generation section 13, an extraction section 14, an output control section 24, a calculation section 21, a determination section 22, and a matching degree calculation section 23. The acquisition section 11, the generation section 12, the query generation section 13, the extraction section 14, the output control section 24, the calculation section 21, the determination section 22, and the matching degree calculation section 23 are configured to realize acquisition means, generation means, query generation means, extraction means, output means, calculation means, determination means, and matching degree calculation means, respectively, in the present example embodiment.

The acquisition section 11 acquires data provided by the input section 25. As an example, the acquisition section 11 may acquire a string. The acquisition section 11 stores the acquired string in the storage section 27.

The generation section 12 generates a result obtained by carrying out the language processing on the string. As an example, the generation section 12 generates a result obtained by carrying out the language processing on the string. As described above, the generation section 12 uses the language model M trained to generate a text based on an input text. That is, the generation section 12 inputs the string into the language model M and generates the result outputted from the language model M as a result obtained by carrying out the language processing on the string. The result generated by the generation section 12 is also referred to as the first result. The generation section 12 stores the generated first result, in association with the string, in the storage section 27.

The query generation section 13 generates a query from the first result generated by the generation section 12. Herein, the “query” is a string indicating a query, a request, or the like. The query generation section 13 stores the generated query, in association with the first result, in the storage section 27. The query generation section 13 may generate one query from one first result, or alternatively, may generate multiple queries from one first result.

When the first result generated by the generation section 12 includes a string, the query generation section 13 may generate a query by using a part or the entirety of the string as it is. Further, the query generation section 13 may, for example, extract one or more keywords from the string and use the one or more extracted keywords to generate a query. More specifically, the query generation section 13 may, for example, extract one or more keywords from the string and use a combination of the one or more extracted keywords and a sentence including the one or more keywords, as a query. Further, the query generation section 13 may use, as a query, the one or more keywords, extracted from the string, as they are.

Further, when the first result generated by the generation section 12 includes an image, the query generation section 13 may, for example, detect an object in the image by using a method of detecting an object in an image, and use a string as it is, corresponding to the detected object, as a query. More specifically, for example, the query generation section 13 may use a string indicating the name of an object detected in the image as a query. Examples of the method of detecting an object in an image may include, but not limited to, Faster-RCNN, Single Shot MultiBox Detector (SSD), and You Only Look Once (YOLO).

Further, as an example, the query generation section 13 may cut out one or more parts of the first result (text etc.) generated by the generation section 12, and generate a query for each of the cut parts. The following description will discuss an example of the flow of a process when the first result includes a string. First, the query generation section 13 cuts out multiple spans of a string included in the first result. Herein, the span is a semantic block of sentence(s) and is an example of the “part” used in this specification. The query generation section 13 may, for example, cut out each sentence included in the string included in the first result, as a span. Further, for example, when the first result includes a list in itemized form, the query generation section 13 may cut out each item included in the list as a span. Further, for example, when the first result includes multiple paragraphs, the query generation section 13 may cut out each paragraph as a span. Further, the span generation section 13 may cut out each word included in the string as a span. To each span, the output control section 24 described later adds information identifying a document extracted by the extraction section 14. That is, it can also be said that each span cut out by the query generation section 13 is a unit to which information identifying a document is added.

Next, the query generation section 13 generates queries based on the cut spans. The query generation section 13 may generate one query from one span, or alternatively, may generate multiple queries from one span. The query generation section 13 may use a span as it is as a query, or alternatively, may extract a part of a string included in a span and use the part as it is as a query. Further, the query generation section 13 may extract one or more keywords from each span and use the one or more extracted keywords to generate a query. More specifically, the query generation section 13 may, for example, extract one or more keywords from each span and use the one or more extracted keywords as a query. Further, the query generation section 13 may extract one or more keywords from each span and use a combination of the one or more extracted keywords and a sentence including the one or more keywords, as a query.

When one or more keywords are extracted from each span, the query generation section 13 may, for example, extract a keyword from a span by inputting the span into a model that has been trained by machine learning so as to extract a characteristic keyword from a string inputted as input. Further, the query generation section 13 may extract a keyword from each span by the named entity recognition method. When the named entity recognition method is used, the query generation section 13, for example, estimates a type of each word constituting the query by the named entity recognition method and extracts a word of a specific type (e.g., the name of a person, the name of a country, etc.) as a keyword. Herein, the type of each word indicates a result of categorizing using the named entity classification. Further, the query generation section 13 may, for example, extract, from the words constituting the span, a word having a type other than the specific types, as a keyword.

Further, when the first result outputted from the language model M includes an image, the query generation section 13 may, for example, divide the image outputted from the language model M into two or more regions (parts) and generate a query for each region obtained by dividing the image.

The extraction section 14 extracts a document related to the query from a database in a retrieval process using the query generated by the query generation section 13. Examples of the document extracted by the extraction section 14 may include, but not limited to, text data indicating a string, a file created by a predetermined word processing software, a file in PDF format, and a file in HTML format. As an example, the extraction section 14 may extract a document related to the query from a database stored in the storage section 27. As another example, the extraction section 14 may extract a document related to the query from documents stored in a database connected to the information processing apparatus 2 via the network. An example of the database may be an external database such as online dictionaries, news articles, and social networking services (SNSs). The extraction section 14 stores the extracted document, in association with the query, in the storage section 27.

The method of extracting a document related to the query carried out by the extraction section 14 is not limited. For example, the extraction section 14 may extract a document including the query. The extraction section 14 may extract a document related to the query with use of any existing search engine.

Since the document extracted by the extraction section 14 is a document retrieved with use of the query generated from the first result, which is output of the language model M, it can be said that the document extracted by the extraction section 14 is a document highly relevant to the first result. That is, it can also be said that the document extracted by the extraction section 14 is a document that has a high probability of being the support for the output of the language model M.

The output control section 24 outputs, to the output section 26, a result obtained by adding, to the first result, information identifying the document extracted by the extraction section 14. The output control section 24 corresponds to the output section 15 in the first example embodiment described above. As an example, the output control section 24 outputs a result obtained by adding information identifying the document extracted by the extraction section 14 to the first result generated by the generation section 12. The output control section 24 may add information identifying some of multiple documents extracted by the extraction section 14, to the first result generated by the generation section 12. Examples of the information identifying the document may include the document name, the author name, and the date of publication, and the uniform resource locator (URL) that indicates where the document is stored. Since the document extracted by the extraction section 14 is a document that has a high probability of being the support for the output of the language model M, it can be said that the information identifying the document is source information indicating the source of the language model M. The result outputted by the output control section 24 is also referred to as the second result.

The output control section 24 may output, in addition to the second result, the reliability calculated by the calculation section 21 described later. With this configuration, the output control section 24 can present the reliability of the second result to the user. The output control section 24 may be configured to output the second result as an optimized result, in a case where the determination section 22, described later, has determined that the reliability calculated by the calculation section 21 exceeds the threshold. With this configuration, the output control section 24 can output the highly reliable second result.

Instead of adding, to the first result, information identifying all the documents extracted by the extraction section 14, the output control section 24 may add, to the first result, information identifying some of the documents extracted by the extraction section 14. For example, the output control section 24 may output a second result obtained by adding, to the first result (text etc.) generated by the generation section 12, information identifying a document with a matching degree calculated by the matching degree calculation section 23, described later, and satisfying a predetermined condition, among the documents extracted by the extraction section 14. With this configuration, it is possible to output a document that has a higher probability of being the support for the output of the language model M.

Further, when the query generation section 13 generates a query for each part (span) obtained by cutting out of the first result, the output control section 24 may output both the part and the document extracted with use of the query generated for the part, in association with each other. With this configuration, it is possible to output a document that has a high possibility of being the support for each part (span).

The calculation section 21 calculates the reliability of the result. As an example, the calculation section 21 calculates the reliability of the first result (text etc.) generated by the generation section 12. The calculation section 21 stores the calculated reliability, in association with the first result, in the storage section 27. With this configuration, the calculation section 21 can ascertain to what extent the first result is reliable.

The method of calculating the reliability of the first result carried out by the calculation section 21 is not limited. For example, the calculation section 21 may calculate the reliability of the first result with use of existing techniques. As an example, the calculation section 21 may calculate the reliability of the first result with use of both the first result and the document extracted by the extraction section 14. Examples of a method of calculating the reliability of the first result carried out by the calculation section 21 with use of both the first result and the document extracted by the extraction section 14 may include (a) a method based on inter-word distance, (b) a method based on inter-document distance, or (c) a method based on a learning model.

In a case where the method based on the inter-word distance is used, the calculation section 21 calculates the reliability of the first result based on the inter-word distance between a word included in the first result and a word included in the document. Specifically, the calculation section 21 first calculates the inter-word distance for each combination of a word included in the first result and a word included in the document. The calculation section 21 calculates the reliability of the first result such that the shorter the calculated inter-word distance is, the higher the reliability of the first result is.

In a case where the method based on the inter-document distance is used, the calculation section 21 calculates the reliability of the first result based on the inter-document distance between a sentence included in the first result and a sentence included in the document. Specifically, the calculation section 21 first calculates the inter-document distance between a text included in the first result and a text included in the document. The calculation section 21 calculates the reliability of the first result such that the shorter the calculated inter-document distance is, the higher the reliability of the first result is.

In a case where the method based on the learning model is used, the calculation section 21 uses a learning model that has been learned by machine learning to receive two sentences as input and to output the similarity between the two sentences. In this case, the calculation section 21 inputs, into the learning model, a sentence included in the first result and a sentence included in the document. Then, the calculation section 21 calculates the reliability of the first result such that the higher the degree of the similarity outputted from the learning model is, the higher the reliability of the first result is.

In a case where the extraction section 14 has extracted multiple documents, the calculation section 21 calculates the reliability of the first result for each of the multiple documents. The calculation section 21 may calculate an arithmetic average value of the calculated multiple reliabilities as the reliability of the first result.

As another example, the calculation section 21 may calculate the reliability with use of the first result (text etc.), the document extracted by the extraction section 14, and the string (target text) acquired by the acquisition section 11. For example, the calculation section 21 may calculate the reliability, referring to a result obtained by inputting, into the language model M used by the generation section 12, the first result, the document extracted by the extraction section 14, and the string acquired by the acquisition section 11. As an example, the calculation section 21 acquires, from the language model M, an index indicating how correct the first result obtained with reference to the document is, as a response to the string. The calculation section 21 calculates the reliability of the first result such that the higher the output result is (e.g., the larger value the index is), the higher the reliability of the first result is. With this configuration, the calculation section 21 can suitably calculate the reliability of the first result with use of the language model M that has generated the first result.

The calculation section 21 may input the first result, the document extracted by the extraction section 14, and the string acquired by the acquisition section 11 multiple times into the language model M. In this case, the calculation section 21 may score the result outputted from the language model M for each of the multiple times of input (likelihood, positive frequency, etc.), and calculate the reliability of the first result such that the higher the score is, the higher the reliability of the first result is.

Further, instead of the string acquired by the acquisition section 11, the calculation section 21 may input a string instructing to answer whether the first result is correct or not into the language model, considering the document identified by the information added by the output control section 24. In this case also, the calculation section 21 calculates the reliability of the first result such that the more positive the output result is, the higher the reliability of the first result is.

The determination section 22 determines whether the value exceeds a threshold. As an example, the determination section 22 determines whether the reliability calculated by the calculation section 21 exceeds the threshold. The determination section 22 stores the determination result, in association with the reliability, in the storage section 27.

The matching degree calculation section 23 calculates the matching degree between the document extracted by the extraction section 14 and the first result (text etc.) generated by the generation section 12. The matching degree calculation section 23 stores the calculated matching degree, in association with the document, in the storage section 27. In a case where the extraction section 14 has extracted multiple documents, the matching degree calculation section 23 calculates the matching degree for each of the multiple documents. Further, when the query generation section 13 generates a query for each of multiple parts cut out of the first result, the matching degree calculation section 23 calculates the matching degree between the part and the document extracted with use of the query generated for the part. With this configuration, it is possible to output a document that has a high possibility of being the support for each of the multiple parts included in the output of the language model M.

The method of calculating the matching degree carried out by the matching degree calculation section 23 is not limited. As an example, the matching degree calculation section 23 may calculate the matching degree with use of existing techniques. Here, when the first result includes a string, the matching degree calculation section 23 may, for example, calculate the matching degree based on the degree of matching of strings measured by comparing the first result and the document extracted by the extraction section 14. Further, the matching degree calculation section 23 may, for example, carry out a test for entailment on the first result and the document extracted by the extraction section 14, to calculate the matching degree based on the test result.

More specifically, when the first result includes a string, the matching degree calculation section 23 may, for example, calculate the matching degree by (a) a method based on inter-word distance, (b) a method based on inter-document distance, or (c) a method based on a learning model, which will be describe below.

In a case where the method based on the inter-word distance is used, the matching degree calculation section 23 calculates the matching degree based on the inter-word distance between a word included in the string outputted by the generation section 12 and a word included in the document extracted by the extraction section 14. Specifically, first, the matching degree calculation section 23 calculates the inter-word distance for each combination of a word included in the string outputted by the generation section 12 and a word included in the document extracted by the extraction section 14. The matching degree calculation section 23 calculates the matching degree such that the shorter the calculated inter-word distance is, the higher the matching degree is.

In a case where the method based on the inter-document distance is used, the matching degree calculation section 23 calculates the matching degree based on the inter-document distance between a string outputted by the generation section 12 and a document extracted by the extraction section 14. Specifically, first, the matching degree calculation section 23 calculates the inter-document distance between a string outputted by the generation section 12 and a document extracted by the extraction section 14. The matching degree calculation section 23 calculates the matching degree such that the shorter the calculated inter-document distance is, the higher the matching degree is.

In a case where the method based on the learning model is used, the matching degree calculation section 23 uses a learning model that has been learned by machine learning to receive two sentences as input and to output the similarity between the two sentences. In this case, the matching degree calculation section 23 inputs, into the learning model, the string outputted by the generation section 12 and the document extracted by the extraction section 14. Then, the matching degree calculation section 23 calculates the matching degree such that the higher the degree of the similarity outputted from the learning model is, the higher the matching degree is.

As another example, the matching degree calculation section 23 may calculate the matching degree with use of the first result, the document extracted by the extraction section 14, and the string acquired by the acquisition section 11. For example, the matching degree calculation section 23 may calculate the matching degree, referring to a result obtained by inputting, into the language model M used by the generation section 12, the first result, the document extracted by the extraction section 14, and the string acquired by the acquisition section 11. As an example, the matching degree calculation section 23 acquires, from the language model M, an index indicating how correct the first result res1 obtained with reference to the document doc is, as a response to the string str1. The matching degree calculation section 23 calculates the matching degree such that the more positive the output result is (e.g., the greater the value of the index is), the higher the matching degree is. With this configuration, the matching degree calculation section 23 can suitably calculate the matching degree with use of the language model M that has generated the first result.

The matching degree calculation section 23 may input the first result, the document extracted by the extraction section 14, and the string acquired by the acquisition section 11 multiple times into the language model M. In this case, the matching degree calculation section 23 may score the result outputted from the language model M for each of the multiple times of input (likelihood, positive frequency, etc.), and calculate the matching degree such that the higher the score is, the higher the matching degree is.

Further, instead of the string acquired by the acquisition section 11, the matching degree calculation section 23 may input a string instructing to answer whether the first result is correct or not into the language model, considering the document identified by the information added by the output control section 24. In this case as well, the matching degree calculation section 23 calculates the matching degree such that the more positive the output result is, the higher the matching degree is.

When the first result includes an image, the matching degree calculation section 23 may, for example, generate a string from the image and calculate the matching degree between the generated string and the document extracted by the extraction section 14 by any of the abovementioned methods (the method of calculating the distance between words, etc.). An example of the method of generating a string from an image may be, but not limited to, a method of detecting an object in an image and generating a string corresponding to the detected object. Examples of the method of detecting an object in an image may include, but not limited to, Faster-RCNN and You Only Look Once (YOLO).

In the present example embodiment, the matching degree calculation section 23 may calculate the matching degree by a method other than the method of calculating the reliability carried out by the calculation section 21. That is, the reliability and the matching degree may be calculated by different algorithms. For example, the calculation section 21 may calculate the reliability by the abovementioned method (a) that is based on the inter-word distance with use of both the first result and the document extracted by the extraction section 14. Meanwhile, the matching degree calculation section 23 may calculate the matching degree based on a result obtained by inputting, to the language model M, a question sentence including both the first result and the document extracted by the extraction section 14. In this case, an example of the question sentence may be a string indicating the contents of “is the response $ {span} logically proper, considering $ {question} in view of $ {related document}?”. Alternatively, the reliability and the matching degree may be calculated by using language models trained by using different training data.

### (Process Carried Out by Information Processing Apparatus 2)

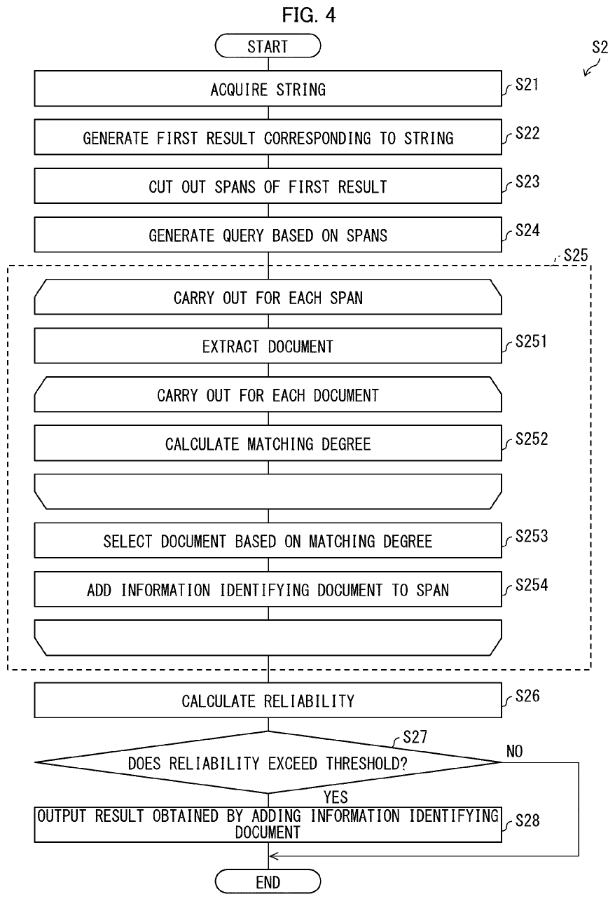

The following description will discuss an information processing method carried out by the information processing apparatus 2, with reference to FIG. 4. FIG. 4 is a flowchart illustrating the flow of an information processing method S2 in accordance with the present example embodiment. An example of FIG. 4 illustrates the flow of a process in a case where the first result outputted by the language model M is a string.

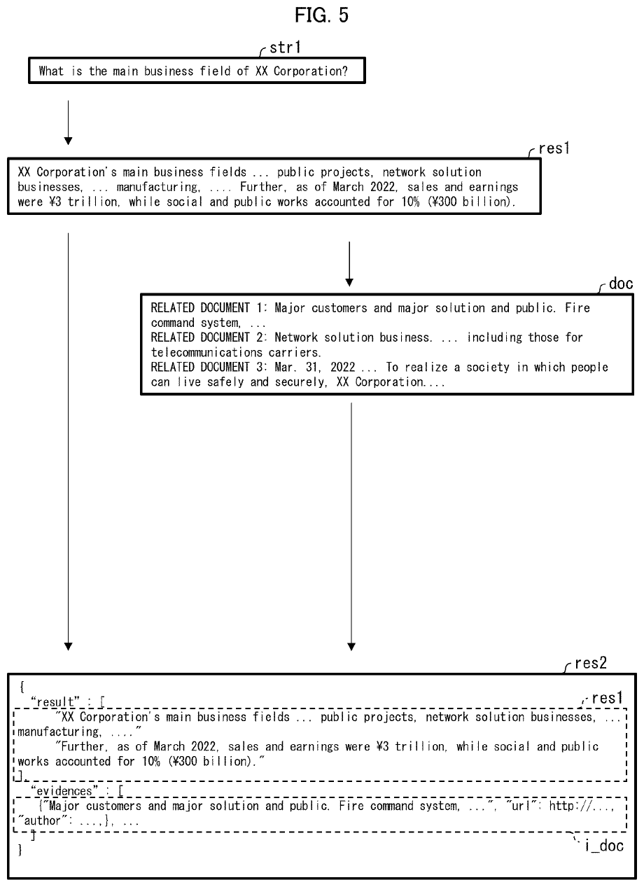

Further, an example of each process carried out by the information processing apparatus 2 will be described with reference to FIG. 5. FIG. 5 is a diagram illustrating an example of each process carried out by the information processing apparatus 2 in accordance with the present embodiment.

### (Step S21)

In step S21, the acquisition section 11 acquires a string. The acquisition section 11 stores the string in the storage section 27. The string acquired by the acquisition section 11 is, for example, a string indicating the contents of a question or an instruction inputted by a user via the input section 25. For example, as illustrated in FIG. 5, the acquisition section 11 acquires a string str1 indicating “what is the main description of business of XX Corporation?”.

### (Step S22)

In step S22, the generation section 12 generates a first result corresponding to the string acquired in step S21 with use of the language model M trained to generate a text based on the input text. The generation section 12 stores the first result, in association with the string, in the storage section 27. For example, as illustrated in FIG. 5, the generation section 12 inputs the string str1 into the language model M, to generate a first result res1 outputted from the language model M.

### (Step S23 and Step S24)

In step S23, the query generation section 13 cuts out multiple spans of the first result generated in step S22. In step S24, the query generation section 13 generates queries based on the cut spans.

### (Step S25)

In step S25, the extraction section 14 extracts a document related to a query in a retrieval process using the query, and the output control section 24 adds, to a span, information identifying the document extracted by the extraction section 14. Step S25 includes steps S251 to S254. Steps S251 to S254 are carried out for each span, which has been cut out by the query generation section 13.

### (Step S251)

In step S251, the extraction section 14 extracts a document related to the query from a database in a retrieval process using the query generated by the query generation section 13. The extraction section 14 stores the extracted document, in association with the query, in the storage section 27. For example, as illustrated in FIG. 5, the extraction section 14 extracts a document doc related to the query generated from the first result res1. The document doc includes multiple documents (“Related Document 1”, “Related Document 2”, “Related Document 3”, etc.).

### (Step S252)

The process in step S252 is carried out for each document extracted by the extraction section 14. In step S252, the matching degree calculation section 23 calculates the matching degree between the span cut out of the first result and the document extracted by using the query generated for the span.

### (Step S253)

In step S253, the output control section 24 selects a document based on the matching degree calculated by the matching degree calculation section 23. That is, the output control section 24 selects a document with a matching degree that satisfies a predetermined condition from among the documents extracted by the extraction section 14. Specifically, the output control section 24 may, for example, select, for each of the multiple spans, a document having a calculated matching degree that exceeds a predetermined threshold. Alternatively, the output control section 24 may select a top-N document or documents (N is a natural number of not less than 1) for each of the multiple spans.

### (Step S254)

In step S254, the output control section 24 adds information identifying the document selected in step S253 for each span to the corresponding span.

### (Step S26)

In step S26, the calculation section 21 calculates the reliability of the first result stored in the storage section 27. The calculation section 21 stores the calculated reliability, in association with the first result, in the storage section 27. For example, as illustrated in FIG. 5, the calculation section 21 calculates the reliability with use of the first result res1, the document doc, and the string str1. As an example, the calculation section 21 inputs the first result res1, the document doc, and the string str1 into the language model M. In this case, the calculation section 21 acquires, from the language model M, an index indicating how correct the first result res1 obtained with reference to the document doc is, as a response to the string str1. The calculation section 21 calculates the reliability with reference to the index.

Further, as an example, instead of the document doc, the calculation section 21 may input a document ($ {1} or $ {3}) included in the first result res1 illustrated in FIG. 5 into the language model M, to calculate the reliability. Further, for example, instead of the string str1, the calculation section 21 may input, into the language model M, the string “can it be said that the first result res1 is correct when considering the information of the document indicated by $ {1}?”, to calculate the reliability.

### (Step S27)

In step S27, the determination section 22 determines whether the reliability stored in the storage section 27 exceeds the threshold.

### (Step S28)

In a case where it has been determined that the reliability exceeds the threshold in step S27 (step S27: YES), then, in step S28, the output control section 24 outputs a result (second result) obtained by adding information identifying the document stored in the storage section 27 to the first result stored in the storage section 27. For example, as illustrated in FIG. 5, the output control section 24 generates a second result res2 obtained by adding information i_doc identifying the document to the first result res1.

The output control section 24 may generate a second result res2 obtained by adding information identifying only a document that has been referred to when the language model M generates the first result res1, among the documents doc extracted by the extraction section 14. For example, as illustrated in FIG. 5, the output control section 24 adds the information i_doc identifying the document indicated by “$ {1}” and the document indicated by “$ {3}” to the first result res1 when the first result res1 includes “$ {1}” and “$ {3}”, to generate the second result res2.

On the other hand, in a case where it has been determined that the reliability does not exceed the threshold in step S27 (step S26: NO), the information processing apparatus 2 terminates the information processing method S2 without causing the output control section 24 to output the second result.

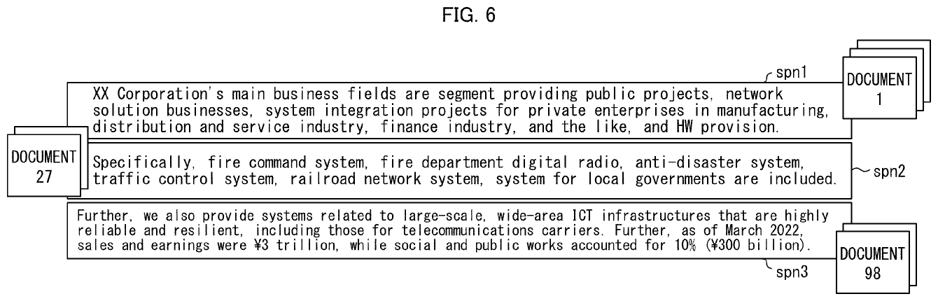

FIG. 6 is a diagram illustrating a specific example of the second result outputted by the output control section 24. As an example, the output control section 24 outputs data indicating the second result to the display device connected to the output section 26, to cause the display device to display an image indicating the second result. In the example of FIG. 6, the second result includes three spans spn1, spn2, and spn3, each of which is associated with information identifying the document extracted by using the query generated for the corresponding span. That is, the output control section 24 outputs the span and the document extracted by using the query generated for the span, in association with each other. The information identifying the document includes, for example, both the document and link information indicating the storage location of the document. In the example of FIG. 6, for example, when the user carries out an operation of selecting a document associated with a span through the input section 25, the output control section 24 may acquire the selected document by using the link information thereof, and display the acquired document on the display device.

### (Example Advantage of Information Processing Apparatus 2)

Thus, the information processing apparatus 2 in accordance with the present example embodiment generates a query from a first result obtained by inputting an acquired string into the language model M, retrieves a document related to the query by using the generated query, and outputs a result obtained by adding information identifying the retrieved document to the first result. With this configuration, the information processing apparatus 2 can output information that identifies a document having a high probability of being the support for the output of the language model M, together with the output result of the language model M.

Thus, the information processing apparatus 2 can present, to the user, both the output of the language model M and the information on the document having a high probability of being the support of the output, so that the user can check the contents of the document and the information on the document, to facilitate determination of whether the output of the language model M is reliable. According to the present example embodiment, it is possible to facilitate determination of the reliability of the output of the language model M.

### Third Example Embodiment

The following will discuss in detail a third example embodiment of the present invention, with reference to drawings. The same reference numerals are given to constituent elements which have functions identical to those described in the above example embodiments, and descriptions as to such constituent elements are omitted as appropriate.

### (Outline and Configuration of Information Processing System 100)

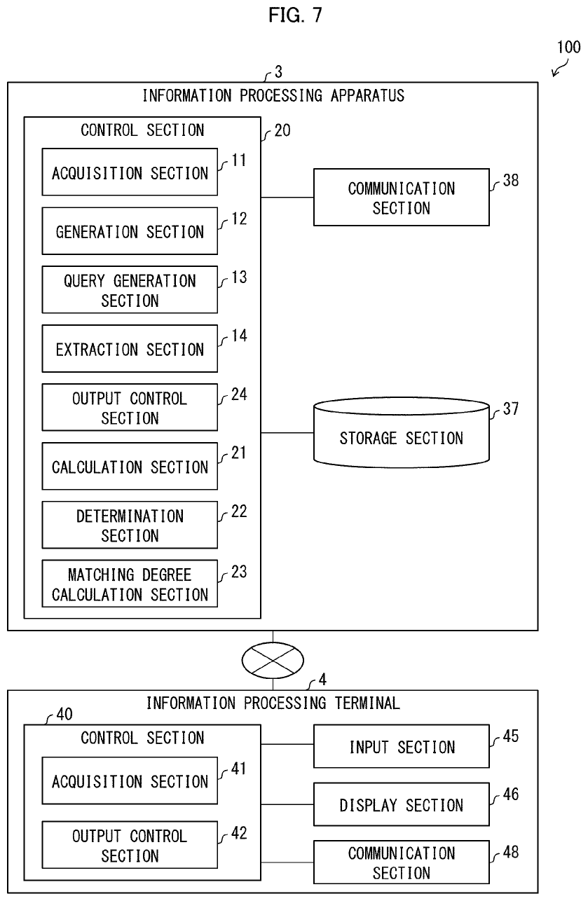

The following description will discuss the outline and the configuration of an information processing system 100 in accordance with the present example embodiment with reference to FIG. 7. FIG. 7 is a block diagram illustrating the configuration of the information processing system 100 in accordance with the present example embodiment.

As illustrated in FIG. 7, the information processing system 100 includes an information processing apparatus 3 and an information processing terminal 4. The information processing apparatus 3 and the information processing terminal 4 are connected via a network so that they can communicate with each other. An example of the information processing apparatus 3 may be a stationary computer. Examples of the information processing terminal 4 may include a smartphone, a tablet terminal, and a laptop computer.

In the information processing system 100, the information processing terminal 4 acquires a target text, and the information processing apparatus 3 carries out language processing on the target text with use of a language model M. Further, the information processing apparatus 3 outputs a result obtained by carrying out the language processing to the information processing terminal 4. Also in the following description, the “target text” and the “text” are also referred to as the “string”.

As an example, in the information processing system 100, the user of the information processing terminal 4 creates an account for using the information processing system 100 and registers the basic information (name, age, gender, email address, etc.). When the user inputs a string related to medical counseling into the information processing terminal 4, the information processing apparatus 3 outputs a response to the medical counseling. Examples of the user input may include symptoms, chronic diseases, and usage conditions of drugs. In response to the medical counseling, the information processing apparatus 3 outputs, in addition to the response, information that supports the response.

### (Configuration of Information Processing Apparatus 3)

As illustrated in FIG. 7, the information processing apparatus 3 includes a control section 20, a storage section 37, and a communication section 38.

The storage section 37 is identical in configuration and function to the storage section 27 described above. Further, the storage section 37 stores documents and information on authors of the documents.

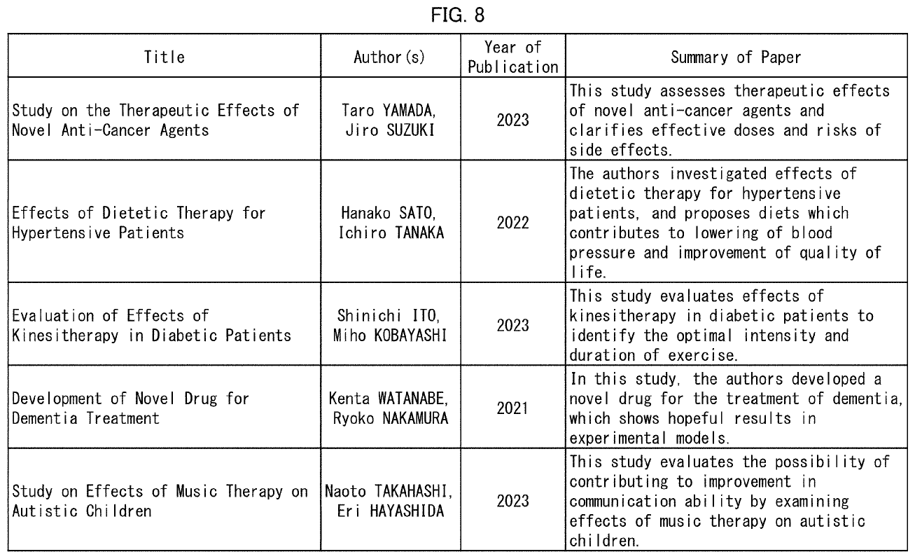

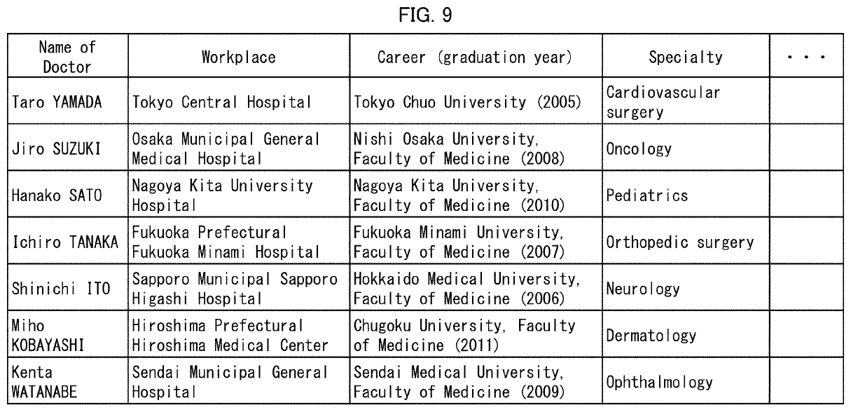

FIGS. 8 and 9 show examples of the information on the documents and authors stored in the storage section 37. FIG. 8 is a table showing an example of the documents in the present example embodiment. FIG. 9 is a table showing an example of the information on the authors of the documents in the present example embodiment. The information on the authors of the documents is merely an example of the information identifying a document in this specification.

As illustrated in FIG. 8, the documents stored in the storage section 37 is those regarding healthcare. As illustrated in FIG. 8, each document stored in the storage section 37 may be stored in such a manner as to be associated with the author or authors, the year of publication, and the abstract of the paper. The information indicating the author or authors of the document, the year of publication, and the abstract of the paper is merely an example of the information identifying a document in this specification.

Further, as illustrated in FIG. 9, information on each author who wrote a document stored in the storage section 37 may be stored in the storage section 37. As illustrated in FIG. 9, as the information on an author who wrote a document, the name of the doctor, the workplace, the career, and the specialty may be stored in the storage section 37.

Thus, the document in the information processing system 100 is a document regarding healthcare and is a document in which the career, specialty, or the like of the author of the document is clear. Therefore, it can be said that the document regarding healthcare in the information processing system 100 is a highly reliable document.

The communication section 38 is an interface for transmitting and receiving data to and from an external apparatus via a network. As an example, the communication section 38 transmits data provided by the control section 20 to the information processing terminal 4 and provides data received from the information processing terminal 4 to the control section 20. Examples of the communication section 38 may include, but not limited to, a communication chip in various communication standards such as Ethernet (registered trademark), Wi-Fi (registered trademark), and radio communications standard for mobile data communications networks, and a USB-compliant connector.

The control section 20 controls constituent elements included in the information processing apparatus 3. Further, as illustrated in FIG. 7, the control section 20 includes an acquisition section 11, a generation section 12, a query generation section 13, an extraction section 14, an output control section 24, a calculation section 21, a determination section 22, and a matching degree calculation section 23. The acquisition section 11, the generation section 12, the query generation section 13, the extraction section 14, the output 21, the control section 24, the calculation section determination section 22, and the matching degree calculation section 23 are configured to realize acquisition means, generation means, query generation means, extraction means, output means, calculation means, determination means, and matching degree calculation means, respectively, in the present example embodiment.

The acquisition section 11 acquires data provided by the communication section 38. As an example, the acquisition section 11 acquires a string. The acquisition section 11 stores the acquired string in the storage section 37.

The generation section 12 generates a first result obtained by carrying out the language processing on the string. The method of generating the first result carried out by the generation section 12 is as described above. The generation section 12 stores the generated first result, in association with the string, in the storage section 37.

The query generation section 13 generates a query from the first result generated by the generation section 12. The method of generating a query carried out by the query generation section 13 is as described above. The query generation section 13 stores the generated query, in association with the first result, in the storage section 37.

The extraction section 14 extracts a document related to the query from a database in a retrieval process using the query generated by the query generation section 13. The method of extracting the document carried out by the extraction section 14 is as described above. Further, the extraction section 14 acquires, from the storage section 37, information on an author of the extracted document. The extraction section 14 stores the information on the extracted document and the author of the extracted document in the storage section 37, in association with the string.

The output control section 24 outputs, to the communication section 38, a second result obtained by adding, to the first result generated by the generation section 12, information identifying the document extracted by the extraction section 14. Further, the output control section 24 may output a result obtained by adding the information on the author of the document extracted by the extraction section 14.

Further, as described above, the output control section 24 may output, in addition to the second result, the reliability calculated by the calculation section 21.

The output control section 24 may be configured to output the second result in a case where the determination section 22 has determined that the reliability calculated by the calculation section 21 exceeds the threshold.

The calculation section 21 calculates the reliability of the result. As an example, the calculation section 21 calculates the reliability of the first result generated by the generation section 12. The method of calculating the reliability of the first result carried out by the calculation section 21 is as described above. The calculation section 21 stores the calculated reliability, in association with the first result, in the storage section 37.

The determination section 22 determines whether the value exceeds a threshold. As an example, the determination section 22 determines whether the reliability calculated by the calculation section 21 exceeds the threshold. The determination section 22 stores the determination result, in association with the reliability, in the storage section 37.

### (Configuration of Information Processing Terminal 4)

As illustrated in FIG. 7, the information processing terminal 4 includes a control section 40, an input section 45, a display section 46, and a communication section 48.

The control section 40 controls constituent elements included in the information processing terminal 4. Further, as illustrated in FIG. 6, the control section 40 includes an acquisition section 41 and an output control section 42.

The acquisition section 41 acquires data provided by the input section 45 or the communication section 48. As an example, the acquisition section 41 acquires a string from the input section 45. As another example, the acquisition section 41 acquires a second result from the communication section 48. The acquisition section 41 provides the acquired data to the output control section 42.

The output control section 42 outputs the data to the display section 46 or the communication section 48. As an example, the output control section 42 outputs the string acquired by the acquisition section 41 to the communication section 48. As another example, the output control section 42 outputs the second result acquired by the acquisition section 41 to the display section 46.

The input section 45 is an interface for receiving data input. Examples of the input section 45 may include a mouse, a keyboard, a touch pad, and a microphone. The input section 45 provides the received data to the control section 40.

The display section 46 is a device for displaying an image. As an example, the display section 46 displays an image based on the data provided by the control section 40. Examples of the display section 46 may include a liquid crystal display and an organic electro luminescence (EL) display.

The communication section 48 is an interface for transmitting and receiving data to and from an external apparatus via a network. As an example, the communication section 48 transmits the data provided by the control section 40 to the information processing apparatus 3 and provides data received from the information processing apparatus 3 to the control section 40. Examples of the communication section 48 may include, but not limited to, a communication chip in various communication standards such as Ethernet, Wi-Fi, and radio communications standard for mobile data communications networks, and a USB-compliant connector.

### (Process Carried Out in Information Processing System 100)

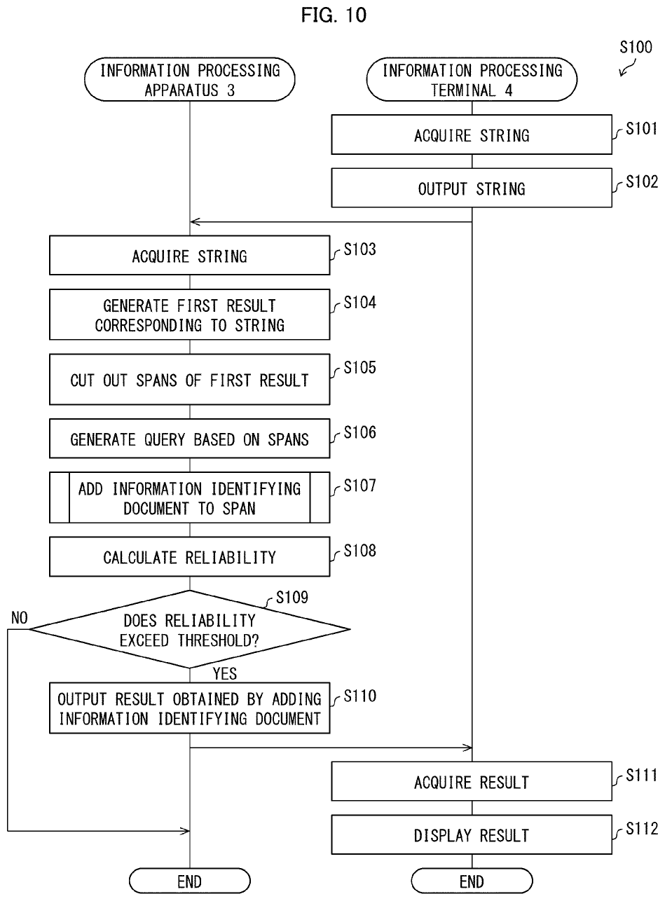

The following description will discuss an information processing method carried out in the information processing system 100, with reference to FIG. 10. FIG. 10 is a flowchart illustrating the flow of an information processing method S100 in accordance with the present example embodiment.

### (Step S101)

In step S101, the acquisition section 41 of the information processing terminal 4 acquires a string. The acquisition section 41 provides the string to the output control section 42.

### (Step S102)

In step S102, the output control section 42 outputs the string provided by the acquisition section 41 to the information processing apparatus 3 via the communication section 48.

### (Step S103)

In step S103, the acquisition section 11 of the information processing apparatus 3 acquires, via the communication section 38, the string outputted from the information processing terminal 4. The acquisition section 11 stores the string in the storage section 37.

### (Step S104)

In step S104, the generation section 12 generates a first result corresponding to the string acquired in step S103 with use of the language model M trained to generate a text based on the input text. The generation section 12 stores the first result, in association with the string, in the storage section 37.

### (Step S105 and Step S106)

In step S105, the query generation section 13 cuts out multiple spans of the first result generated in step S104. In step S106, the query generation section 13 generates queries based on the cut spans.

### (Step S107)

In step S107, the extraction section 14 extracts a document related to the query in a retrieval process using the query. Further, the output control section 24 acquires, from the storage section 37, information on an author of the extracted document. Further, the output control section 24 stores, in the storage section 37, a second result obtained by adding, to the span, both information identifying the document extracted by the extraction section 14 and the information on the author or authors of the document.

### (Step S108)

In step S108, the calculation section 21 calculates the reliability of the first result stored in the storage section 37. The calculation section 21 stores the calculated reliability, in association with the first result, in the storage section 37.

### (Step S109)

In step S109, the determination section 22 determines whether the reliability stored in the storage section 37 exceeds the threshold.

### (Step S110)

In a case where it has been determined that the reliability exceeds the threshold in step S109 (step S109: YES), then, in step S110, the output control section 24 outputs, to the information processing terminal 4, a second result obtained by adding information identifying the document stored in the storage section 37 to the first result stored in the storage section 37.

### (Step S111)

In step S111, the acquisition section 41 of the 4 acquires, via the information processing terminal communication section 48, the second result outputted from the information processing apparatus 3. The acquisition section 41 provides the acquired second result to the output control section 42.

### (Step S112)

In step S112, the output control section 42 displays the second result provided by the acquisition section 41 on the display section 46.

On the other hand, in a case where it has been determined that the reliability does not exceed the threshold in step S109 (step S109: NO), the information processing system 100 terminates the information processing method S100 without causing the output control section 24 to output the second result.

Alternatively, the output control section 24 may output, to the information processing terminal 4, information indicating that no highly reliable result could be generated, via the communication section 38. In this case, the output control section 42 of the information processing terminal 4 may display on the display section 46 that no reliable result could be generated.

### (First Example of Image Displayed by Information Processing Terminal 4)

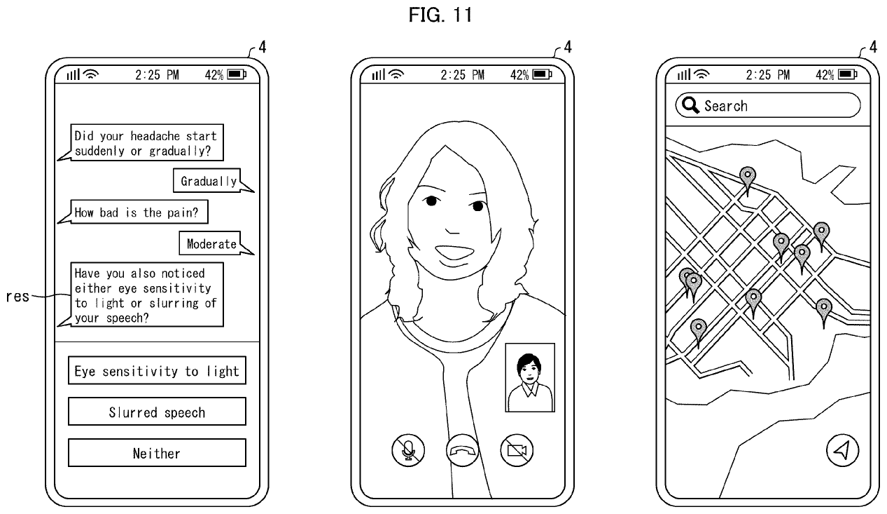

An example of the image displayed by the information processing terminal 4 will be described with reference to FIG. 11. FIG. 11 is a diagram illustrating an example of images displayed on the information processing terminal 4 in accordance with the present example embodiment.

As described above, the output control section 42 of the information processing terminal 4 displays the second result on the display section 46. As an example, as illustrated at the left diagram of FIG. 11, the output control section 42 displays the second result res, that is, “Have you also noticed either eye sensitivity to light or slurring of your speech?”, in response to the input, “Moderate”, which indicates a symptom and is inputted by the user. Further, in this example, the output control section 42 displays, on the display section 46, the information identifying the document retrieved by using the query generated from the second result res, in addition to the second result res. When the user carries out an operation of selecting information identifying a document through the image displayed on the display section 46, the information processing terminal 4 may carry out a process of acquiring information of the document corresponding to the selected information, to display the information.

Further, when the acquisition section 41 acquires information indicating an operation indicating that the user selects the second result res (e.g., an operation performed by touching a portion in the display section 46 at which the second result res is displayed), the output control section 42 may display information identifying the document. Further, as described above, a case where the output control section 24 of the information processing apparatus 3 outputs the second result obtained by adding information on the author of the document, the output control section 42 may display information on the author of the document in addition to the information identifying the document.

### (Second Example of Image Displayed by Information Processing Terminal 4)

In step S110 described above, the output control section 24 may output, in addition to the second result, information for communicating with a doctor, to the information processing terminal 4. As an example, the output control section 24 may refer to the information identifying the document and the information on the author of the document, and then may output the contact information of a doctor in the same professional field as that of the author to the information processing terminal 4, in addition to the second result.

For example, when the output control section 24 outputs the doctor's contact information to the information processing terminal 4 in addition to the second result in the abovementioned step S110, the information processing terminal 4 displays the doctor's contact information on the display section 46 in addition to the second result. In this configuration, when the acquisition section 41 acquires information indicating an operation indicating that the user selects the doctor's contact information (e.g., an operation performed by touching a portion in the display section 46 at which the doctor's contact information is displayed), the output control section 42 makes a call to the doctor using the contact information.

With this configuration, the information processing terminal 4 enables the user to make a video call with the doctor as illustrated in the central diagram in FIG. 11.

### (Third Example of Image Displayed by Information Processing Terminal 4)

In step S110 described above, the output control section 24 may output, to the information processing terminal 4, information on an action proposed to the user, in addition to the second result. Examples of actions proposed to the user may include going to a hospital and obtaining a doctor's prescription medicine. The following description will discuss a configuration in which the output control section 24 outputs, to the information processing terminal 4, information indicating a method of obtaining a doctor's prescription medicine.

In a case where the second result includes information identifying a medicine, the output control section 24 may output, to the information processing terminal 4, the place of a pharmacy where the medicine is sold, in addition to the second result.

Alternatively, as described above, after the user communicates with the doctor, the doctor may output a prescription for the user to the information processing terminal 4. When the user obtains the prescription, the information processing terminal 4 may output, in addition to the second result, to the information processing terminal 4, the place of a pharmacy where the medicine is sold, described in the prescription.

With this configuration, as illustrated in the right diagram in FIG. 11, the information processing terminal 4 can present the user with a place where the user can purchase the medicine prescribed by the doctor.

### (First Example Advantage of Information Processing System 100)

Thus, in the information processing system 100 in accordance with the present example embodiment, the information processing apparatus 3 generates a query from a first result obtained by inputting a string acquired by means of the information processing terminal 4 into the language model M, retrieves a document related to the query by using the generated query, and outputs a result obtained by adding information identifying the retrieved document to the first result. With this configuration, the information processing apparatus 3 can output information that identifies a document having a high probability of being the support for the output of the language model M, together with the output result of the language model M.

Thus, since the information processing apparatus 3 can present, to the user, both the output of the language model M and the information identifying the document having a high probability of being the support of the output, the user can check the information present to the user; this facilitates user's determination of whether the output of the language model M is reliable. According to the present example embodiment, it is possible to present, to a user, information for determining whether the output is reliable, besides the output of the language model M.

### (Second Example Advantage of Information Processing System 100)

In the information processing system 100, the information processing terminal 4 take a question in medical counseling, and the information processing apparatus 3 generates a response to the medical counseling. The following description will discuss an example advantage achieved in a configuration in which the information processing system 100 outputs a response to medical counseling of the user.

First, in the information processing system 100, the information processing apparatus 3 generates a response to the medical counseling with use of the language model M. In the configuration, the user can take medical counseling via the information processing terminal 4 such as, for example, a smartphone. Therefore, with the information processing system 100, the user can easily take medical counseling. Further, with the information processing system 100, the user can easily take medical counseling anytime and anywhere. Further, with the information processing system 100, for example, the user can receive general information and/or advice about symptoms at a stage before the user receives any diagnosis or treatment.

Further, since the information processing terminal 4 allows the user to ask medical counseling therethrough and outputs a response, the user can take medical counseling without going to the hospital. Further, the information processing system 100 allows the user to obtain a response to medical counseling more quickly than when the user go to the hospital. Further, in the information processing system 100, response to the medical counseling can be provided without intervention by a doctor, resulting in a decrease in cost of responding to medical counseling.

### [Software Implementation Example]

Some or all of the functions of the information processing apparatuses 1, 2, and 3 may be realized by hardware such as an integrated circuit (IC chip) or may be alternatively realized by software.

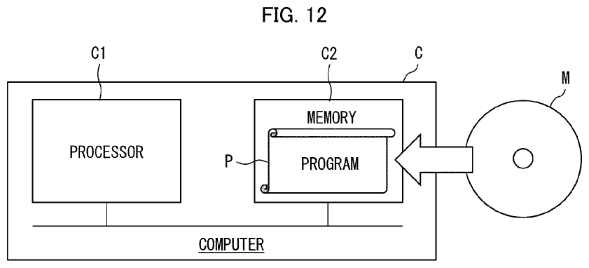

In the latter case, the information processing apparatuses 1, 2, and 3 are each realized by, for example, a computer that executes instructions of a program that is software realizing the functions. FIG. 12 illustrates an example of such a computer (hereinafter referred to as “computer C”). The computer C includes at least one processor C1 and at least one memory C2. The memory C2 stores a program P for causing the computer C to operate as each of the information processing apparatuses 1, 2, and 3. In the computer C, the functions of each of the information processing apparatuses 1, 2, and 3 are realized by the processor C1 reading the program P from the memory C2 and executing the program P.

The processor C1 may be, for example, a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination thereof. The memory C2 may be, for example, a flash memory, a hard disk drive (HDD), a solid state drive (SSD), or a combination thereof.

Note that the computer C may further include a random access memory (RAM) in which the program P is loaded when the program P is executed and/or in which various kinds of data are temporarily stored. The computer C may further include a communication interface for transmitting and receiving data to and from another apparatus. The computer C may further include an input/output interface for connecting the computer C to an input/output apparatus(es) such as a keyboard, a mouse, a display and/or a printer.

The program P can be recorded in a non-transitory tangible storage medium M from which the computer C can read the program P. Such a storage medium M may be, for example, a tape, a disk, a card, a semiconductor memory, a programmable logic circuit, or the like. The computer C can acquire the program P via the storage medium M. The program P can be transmitted via a transmission medium. The transmission medium may be, for example, a communications network, a broadcast wave, or the like. The computer C can acquire the program P also via such a transmission medium.

### [Additional Remark 1]

The present invention is not limited to the foregoing example embodiments, but may be altered in various ways by a skilled person within the scope of the claims. For example, the present invention also encompasses, in its technical scope, any example embodiment derived by appropriately combining technical means disclosed in the foregoing example embodiments.

### [Additional Remark 2]

Some or all of the above example embodiments can be described as below. Note, however, that the present invention is not limited to example aspects described below.

The whole or part of the example embodiments disclosed above can be described as, but not limited to, the following supplementary notes.

### (Supplementary Note 1)

An information processing apparatus includes: acquisition means for acquiring a target text; generation means for generating a text corresponding to the acquired target text with use of a language model trained to generate a text based on an input text; query generation means for generating a query from the text generated by the generation means; extraction means for extracting a document related to the query from a database in a retrieval process using the query; and output means for outputting a result obtained by adding information identifying the document to the text generated by the generation means.

### (Supplementary Note 2)

The information processing apparatus according to Supplementary note 1, further including calculation means for calculating reliability of the text generated by the generation means.

### (Supplementary Note 3)

The information processing apparatus according to Supplementary note 2, further including determination means for determining whether the reliability exceeds a threshold, wherein, in a case where the determination means has determined that the reliability exceeds the threshold, the output means outputs the result obtained by adding the information identifying the document as an optimized result.

### (Supplementary Note 4)

The information processing apparatus according to Supplementary note 2 or 3, wherein the output means outputs the reliability in addition to the result obtained by adding the information identifying the document.

### (Supplementary Note 5)

The information processing apparatus according to any one of Supplementary notes 2 to 4, wherein the calculation means calculates the reliability with use of the text generated by the generation means, the document extracted by the extraction means, and the target text acquired by the acquisition means.

### (Supplementary Note 6)

The information processing apparatus according to any one of Supplementary notes 1 to 5, further including matching degree calculation means for calculating a matching degree between the document extracted by the extraction means and the text generated by the generation means, wherein the output means outputs a result obtained by adding, to the text generated by the generation means, information identifying a document with a matching degree satisfying a predetermined condition, among documents extracted by the extraction means.

### (Supplementary Note 7)

The information processing apparatus according to Supplementary note 6, wherein the query generation means cuts out one or more parts of the text generated by the generation means and generates a query for each of the cut parts, and the matching degree calculation means calculates the matching degree between a part of the parts and the document extracted with use of the query generated for the part.

### (Supplementary Note 8)

The information processing apparatus according to Supplementary note 7, wherein the output means outputs the part and the document extracted with use of the query generated for the part, in association with each other.

### (Supplementary Note 9)

An information processing method including: acquiring, by at least one processor, a target text; generating, by the at least one processor, a text corresponding to the target text with use of a language model trained to generate a text based on an input text; generating, by the at least one processor, a query from the generated text; extracting, by the at least one processor, a document related to the query from a database in a retrieval process using the query; and outputting, by the at least one processor, a result obtained by adding information identifying the document to the generated text.

### (Supplementary Note 10)

A program for causing a computer to carry out: an acquisition process of acquiring a target text; a generation process of generating a text corresponding to the target text with use of a language model trained to generate a text based on an input text; a query generation process of generating a query from the text generated in the generation process; an extraction process of extracting a document related to the query from a database in a retrieval process using the query; and an output process of outputting a result obtained by adding information identifying the document to the text generated in the generation process.

### [Additional Remark 3]

Some or all of the above example embodiments can also be described as below.

### (Supplementary Note 1)

An information processing apparatus including at least one processor, the at least one processor carrying out: an acquisition process of acquiring a target text; a generation process of generating a text corresponding to the acquired target text with use of a language model trained to generate a text based on an input text; a query generation process of generating a query from the text generated in the generation process; an extraction process of extracting a document related to the query from a database in a retrieval process using the query; and an output process of outputting a result obtained by adding information identifying the document to the text generated in the generation process.

### (Supplementary Note 2)

The information processing apparatus according to Supplementary note 1, wherein the processor further carrying out a calculation process of calculating reliability of the text generated in the generation process.

### (Supplementary Note 3)

The information processing apparatus according to Supplementary note 2, wherein the processor further carrying out a determination process of determining whether the reliability exceeds a threshold, wherein in a case where it has been determined, in the determination process, that the reliability exceeds the threshold, the result obtained by adding the information identifying the document is outputted as an optimized result in the output process.

### (Supplementary Note 4)

The information processing apparatus according to Supplementary note 2 or 3, wherein, in the output process, the processor outputs the reliability in addition to the result obtained by adding the information identifying the document.

### (Supplementary Note 5)

The information processing apparatus according to any one of Supplementary notes 2 to 4, wherein, in the calculation process, the processor calculates the reliability with use of the generated in the generation process, the document text extracted in the extraction process, and the target text acquired in the acquisition process.

### (Supplementary Note 6)

The information processing apparatus according to any one of Supplementary notes 1 to 5, the processor further carrying out a matching degree calculation process of calculating a matching degree between the document extracted in the extraction process and the text generated in the generation process, wherein in the output process, the processor outputs a result obtained by adding, to the text generated in the generation process, information identifying a document with a matching degree satisfying a predetermined condition, among the documents extracted in the extraction process.

### (Supplementary Note 7)

The information processing apparatus according to Supplementary note 6, wherein: in the query generation, the processor cuts out one or more parts of the text generated in the generation process and generates a query for each of the cut parts; and, in the matching degree calculation process, the processor calculates the matching degree between a part of the parts and the document extracted with use of the query generated for the part.

### (Supplementary Note 8)

The information processing apparatus according to Supplementary note 7, wherein in the output process, the processor outputs the part and the document extracted with use of the query generated for the part, in association with each other.

### (Supplementary Note 9)

An information processing method including: acquiring, by at least one processor, a target text; generating, by the at least one processor, a text corresponding to the target text with use of a language model trained to generate a text based on an input text; generating, by the at least one processor, a query from the generated text; extracting, by the at least one processor, a document related to the query from a database in a retrieval process using the query; and outputting, by the at least one processor, a result obtained by adding information identifying the document to the generated text.

### (Supplementary Note 10)

Note that the information processing apparatus may further include a memory, and this memory may include a program for causing the processor to carry out the acquisition process, the generation process, the query generation process, the extraction process, and the output process. The program may be stored in a computer-readable, non-transitory, tangible storage medium.

### REFERENCE SIGNS LIST

1, 2, 3 Information processing apparatus
4 Information processing terminal
11, 41 Acquisition section
12 Generation section
13 Query generation section
14 Extraction section
15, 26 Output section
20, 40 Control section
21 Calculation section
22 Determination section
24, 42 Output control section
23 Matching degree calculation section
25, 45 Input section
27, 37 Storage section
38, 48 Communication section
46 Display section

What is claimed is:

1. An information processing apparatus comprising at least one processor, the at least one processor carrying out:
an acquisition process of acquiring a target text;
a generation process of generating a text corresponding to the acquired target text with use of a machine learning model trained to generate a text based on an input text;
a query generation process of generating a query from the text generated in the generation process;
an extraction process of extracting a document related to the query from a database in a retrieval process using the query; and
an output process of outputting a result obtained by adding information identifying the document to the text generated in the generation process.

2. The information processing apparatus according to claim 1, wherein the at least one processor further carries out a calculation process of calculating reliability of the text generated in the generation process.

3. The information processing apparatus according to claim 2, wherein:
the at least one processor further carries out a determination process of determining whether the reliability exceeds a threshold; and
in a case where it has been determined, in the determination process, that the reliability exceeds the threshold, the at least one processor outputs, in the output process, the result obtained by adding the information identifying the document as an optimized result.

4. The information processing apparatus according to claim 2, wherein, in the output process, the at least one processor outputs the reliability in addition to the result obtained by adding the information identifying the document.

5. The information processing apparatus according to claim 2, wherein, in the calculation process, the at least one processor calculates the reliability with use of the text generated in the generation process, the document extracted in the extraction process, and the target text acquired in the acquisition process.

6. The information processing apparatus according to claim 1, wherein:
the at least one processor further carries out a matching degree calculation process of calculating a matching degree between the document extracted in the extraction process and the text generated in the generation process; and
in the output process, the at least one processor outputs a result obtained by adding, to the text generated in the generation process, information identifying a document with a matching degree satisfying a predetermined condition, among documents extracted in the extraction process.

7. The information processing apparatus according to claim 6, wherein;
in the query generation process, the at least one processor cuts out one or more parts of the text generated in the generation process and generates a query for each of the cut parts; and
in the matching degree calculation process, the at least one processor calculates the matching degree between a part of the parts and the document extracted with use of the query generated for the part.

8. The information processing apparatus according to claim 7, wherein, in the output process, the at least one processor outputs the part and the document extracted with use of the query generated for the part, in association with each other.

9. An information processing method comprising:
acquiring, by at least one processor, a target text;
generating, by the at least one processor, a text corresponding to the target text with use of a machine learning model trained to generate a text based on an input text;
generating, by the at least one processor, a query from the generated text;
extracting, by the at least one processor, a document related to the query from a database in a retrieval process using the query; and
outputting, by the at least one processor, a result obtained by adding information identifying the document to the generated text.

10. A computer-readable non-transitory storage medium storing a program for causing a computer to function as an information processing apparatus, the program causing the computer to carry out:
an acquisition process of acquiring a target text;
a generation process of generating a text corresponding to the target text with use of a machine learning model trained to generate a text based on an input text;
a query generation process of generating a query from the text generated in the generation process;
an extraction process of extracting a document related to the query from a database in a retrieval process using the query; and
an output process of outputting a result obtained by adding information identifying the document to the text generated in the generation process.
